(19) United States
(12) Patent Application Publication
(10) Pub. No.: US 2016/0187321 A1
(43) Pub. Date: Jun. 30, 2016

(54) INDUCED EXPRESSION OF PROTEINS IN INSECT CELLS

(71) Applicant: UNIVERSITY OF FLORIDA RESEARCH FOUNDATION, INC., GAINESVILLE, FL (US)

(72) Inventors: JEFFREY RICHARD BLOOMQUIST, MICANOPY, FL (US); FAN TONG, GAINESVILLE, FL (US); LACEY JO JENSON, GAINESVILLE, FL (US)

(21) Appl. No.: 15/062,055

(22) Filed: Mar. 5, 2016

Related U.S. Application Data
(63) Continuation-in-part of application No. PCT/US2014/054433, filed on Sep. 6, 2014.
(60) Provisional application No. 61/874,726, filed on Sep. 6, 2013.

(51) Int. Cl. G01N 33/50; C12P 21/00; C12N 5/07

(52) U.S. Cl. CPC G01N 33/5023; G01N 33/5044; C12N 5/0601; C12P 21/00; G01N 2333/43552; G01N 2333/43591

(57) ABSTRACT

The present invention concerns insect cells that have been induced to express one or more proteins by contact with one or more insect hormones or one or more modulators of the G protein-coupled receptor signaling pathway, compositions comprising such insect cells, a method for inducing protein expression in insect cells, and a screening method using such insect cells.

### CROSS-REFERENCE TO RELATED APPLICATIONS

The present application is a continuation-in-part of International Application Number PCT/US2014/054433, filed Sep. 6, 2014, which claims the benefit of U.S. Provisional Application Ser. No. 61/874,726, filed Sep. 6, 2013, which is hereby incorporated by reference herein in its entirety, including any figures, tables, nucleic acid sequences, amino acid sequences, or drawings.

### GOVERNMENT SUPPORT

This invention was made with government support under grant number 58-0208-0-068 awarded by the USDA Specific Cooperative Agreement. The government has certain rights in the invention.

### BACKGROUND OF THE INVENTION

Insect cells may be used for research of physiology, histology, embryology, molecular biology, pathology and insect virology, and for the production of recombinant proteins. Continuous cell lines have been established from over 100 insect species since the establishment of the first insect cell lines [8]. However, genetically engineering cells is time-consuming and costly.

### BRIEF SUMMARY OF THE INVENTION

The present invention concerns insect cells that have been induced to express one or more proteins by contact with one or more insect hormones or modulators of the G protein-coupled receptor (GPCR) signaling pathway, compositions comprising such insect cells, a method for inducing protein expression in insect cells, and a screening method using such insect cells.

Treatment of immortal insect cell lines with the insect hormone 20-hydroxyecdysone (20-HE) induces expression of insecticide target proteins (e.g., the Kv2 potassium channel) without the need for genetic engineering. These hormone-treated cells may be used for high-throughput screening applications. The invention may be used for insecticide discovery or basic research. Optionally, cells may be made to produce proteins of interest simply by adding an insect hormone or modulator of the GPCR signaling pathway to the growth medium, avoiding the expense of genetically engineered cells.

### BRIEF DESCRIPTION OF THE DRAWINGS

FIGS. 1A and 1B. Anopheles gambiae (Sua1B) cells. FIG. 1A: Sua1B cells in complete Schneiders' medium. FIG. 1B: Sua1B cells in medium with 42 μM 20-HE 48 hours after passage (cell loss).

FIG. 2. Time dependence of 20-HE (42 μM)-induced K⁺ channel expression (thallium fluorescence) in Sua1B cells. Bars are means ±SEM. Statistical analysis was one-way ANOVA with Student-Newman-Keuls post test (P<0.05). Bars labeled by different letters indicate statistical significance.

FIGS. 3A and 3B. 20-HE treatment for 3 hours induces delayed-rectifier (Kv2) K⁺ channel currents in Sua1B cells. FIG. 3A: Whole cell patch clamp trace of treated Sua1B cells showing a delayed rectifier K⁺ channel current. FIG. 3B: Current-voltage relationship of currents shown in FIG. 3A.

FIG. 4. AgKv2 K⁺ channel expression in Sua1B cells treated with 42 μM 20-HE for 3 hours compared to AgKv2 channels transiently expressed in mammalian HEK293 cells. Bars are means ±SEM. Statistical analysis was one-way ANOVA and a Student-Newman-Keuls post test for the Sua1B cells (P<0.05). HEK cell data were analyzed by paired t-test (P value=0.02). Bars labeled by different letters indicate statistical significance.

FIGS. 5A-5F. Sua1B potassium current inhibition with tetraethylammonium (TEA). FIG. 5A: Whole cell patch clamp traces of Sua1B cells showing a delayed rectifier K⁺ channel current. FIGS. 5B-5D: Inhibition of K⁺ current by TEA (0.3-30 mM). FIG. 5E: Current-voltage relationship of currents in FIGS. 5A-5D and their inhibition with TEA. FIG. 5F: Concentration-response curve of TEA on Sua1B cells. Symbols are means ±SEM.

FIGS. 6A-6F. Comparison of native HEK293 K⁺ channels with AgKv2 K⁺ channels expressed in HEK293 cells. Comparison of endogenous potassium currents in mammalian HEK293 cells with AgKv2 expressed in these cells. FIGS. 6A-6B: Whole cell patch clamp traces and current-voltage plots of native currents, and FIG. 6C: engineered AgKv2 under tetracycline (TC) control. FIG. 6D: Inhibition of potassium current by TEA in HEK293 cells expressing the AgKv2 gene. FIG. 6E: Concentration-response curves of TEA inhibition of K+ currents in HEK cells with and without TC. FIG. 6F: Comparison of maximal current amplitudes at +100 mV in the presence and absence of TC. Bars are means ±SEM.

FIGS. 7A and 7B. It is known that thallium ions can traverse potassium ion channels. FIG. 7A: Induction of potassium channels in Sua12b cells by 20-HE, and the blocking action of 4-aminopyridine (4-AP) on thallium ion uptake (as shown in FIG. 4) is specific, since there is no effect of the sodium channel blocking agent, tetrodotoxin. FIG. 7B: 3 hours of 20-HE treatment of Anopheles gambiae Sua1b cells. The left panel of FIG. 7B shows that imidacloprid (IMI), an insecticide that activates nicotinic acetylcholine receptors, increases calcium fluorescence above controls in Sua12b cells. Moreover, this increased fluorescence is blocked by 100 μM mecamylamine (MML), a specific nicotinic receptor blocker. In the absence of 20-HE (right panel), there is no imidacloprid effect.

FIGS. 8A-8C. FIG. 8A: 6 hours of exposure to 20-HE enhances expression of nicotinic acetylcholine receptors in lepidopteran Sf21 cells, and that there may be some background expression of this receptor in these cells. The left panel of FIG. 8A shows a greater imidacloprid-induced calcium fluorescence (10 μM imidacloprid=IMI) compared to matched controls (Ctl). Imidacloprid-induced calcium fluorescence is blocked by 100 μM mecamylamine (MML). In cells without 20-HE, there is a reduced effect of imidacloprid, and a variable and not statistically significant reduction by mecamylamine. FIG. 8B: Response of a patch clamped Sf21 cell to imidacloprid treatment after 6 hours in 20-HE. The inward current is consistent with the fluorescence results with imidacloprid in FIG. 8A. FIG. 8C: Sf21 cells also respond to 3 hours treatment with 20-E by expressing outward potassium currents similar to Kv2 currents it induced in Sua-1b cells (FIG. 3B). The current voltage plot was generated by subtracting TEA-insensitive current from total current at each voltage.

FIG. 9. FIG. 9 shows that caffeine alone has approximately the same effect as caffeine and 20-HE for expressing imdacloprid sensitivity. FIG. 9 shows the results of an experiment comparing the effect of vehicle and 10 μM imidacloprid (IMI.) after 6 hours exposure of Sf21 cells to caffeine (right side of FIG. 9) or caffeine +20-HE (left side of FIG. 9). Expression of IMI-induced fluorescence is the same, with even lower vehicle background with caffeine, at least in this particular experiment.

FIG. 10. FIG. 10 shows that bethanechol, a muscarinic agonist and mimic of acetylcholine, preferentially activates the muscarinic-type receptor, increases calcium fluorescence in Sua1B cells treated with 20-HE. The response was a typical sigmoid curve with an EC₅₀ (concentration where the response is half maximal) of 0.25 micromolar. In the absence of the hormone, bethanechol activated fluorescence, but had a less potent effect, with an EC₅₀ of 48 micromolar.

### DETAILED DESCRIPTION OF THE INVENTION

The present invention concerns an insect cell (one or more insect cells) that has been induced to express one or more proteins by contact with one or more insect hormones and/or one or more modulators of the G protein-coupled receptor (GPCR) signaling pathway (also referred to herein as a “GPCR signaling pathway modulator”, “GPCR signaling modulator” or “GPCR modulator”). The cells may be isolated or purified using techniques known in the art.

Another aspect of the invention concerns a composition comprising an insect cell that has been induced to express one or more proteins by contact with one or more insect hormones and/or GPCR signaling pathway modulators.

Another aspect of the invention concerns an insect cell and one or more insect hormones and/or one or more GPCR signaling pathway modulators. In some embodiments, the insect cell has been induced to express one or more proteins by the one or more insect hormones or GPCR signaling pathway modulators.

In some embodiments, the composition of the invention is a cell culture and may include cell culture medium (e.g., growth media), and may be contained within a culture vessel.

Another aspect of the invention concerns a method for inducing protein expression in insect cells, comprising contacting an insect cell with an effective amount of one or more insect hormones or GPCR signaling pathway modulators to induce expression of one or more proteins. In some embodiments, the insect cell is in cell culture with a culture medium, and wherein the one or more insect hormones or GPCR signaling pathway modulators are added to the culture medium (e.g., growth media). The term “contacting” in this context is intended to mean bringing the insect cell and the one or more insect hormones and/or GPCR signaling pathway modulators into contact, whether the cell is physically brought into contact with the hormone or GPCR signaling pathway modulator, or vice-versa, or the insect cell and hormone or GPCR signaling pathway modulator are brought into contact with each other.

The insect hormone or GPCR signaling pathway modulator and the insect cell may be brought into contact, for example, by bringing an isolated insect hormone or isolated GPCR signaling modulator into contact. The insect hormone or GPCR signaling pathway modulator may be in isolated form when contact is made, or the insect hormone or GPCR signaling pathway modulator may be a component in a composition, and the composition and insect cell are brought into contact. In some embodiments, the insect hormone or GPCR signaling pathway modulator is added or otherwise included in a culture medium for the insect cell. In some embodiments, cells producing the insect hormone or GPCR signaling pathway modulator are added or otherwise included in a culture medium for the insect cell (e.g., as a co-culture). Any method or procedure that brings the insect cell and insect hormone or GPCR signaling pathway modulator into contact to induce protein expression may be utilized.

The insect cell and insect hormone or GPCR signaling pathway modulator can be brought into contact on a substrate such as a culture vessel or sample plate. However, the precise form of the culture is not crucial and may be any that is known to those of skill in the art. For example, the cells may be adhered to the inside surface of a vessel in a monolayer, suspended in solution, or grown in a manner that allows three-dimensional growth in the culture, e.g., on three-dimensional “scaffolding”, etc.

In some embodiments, the protein expression method includes characterizing the one or more expressed proteins (e.g., characterizing the proteins as insecticide target protein (a protein targeted by an insecticide for the insecticide's mechanism of action) or a protein that is not an insecticide target protein).

Another aspect of the invention is a screening method, comprising: providing an insect cell, wherein the insect cell has been contacted with an effective amount of one or more insect hormones or GPCR signaling pathway modulators to express one or more proteins; contacting the insect cell with a substance; and evaluating one or more parameters to determine the effect of the substance on the insect cell.

Another aspect of the invention is a screening method, comprising contacting an insect cell with an effective amount of one or more insect hormones or GPCR signaling pathway modulators; contacting the insect cell with the substance before, during, or after contact with the one or more insect hormones or GPCR signaling pathway modulators; and evaluating one or more parameters to determine the effect of the substance on the insect cell.

In the screening methods of the invention, various parameters may be assessed to determine the effect of the substance on the insect cell. These parameters may include, for example, cell survival, growth, internal pH, respiration, uptake or release of metabolites or nutrients, ion uptake or release via fluorescence, radioisotopic or other methods, or ion current measurements via electrophysiology. Various in vitro assays may be utilized. For example, the evaluation may comprise measuring the function of one or more ion channels before, during, and/or after contact with the substance and/or before, during, and/or after contact with the one or more insect hormones or GPCR signaling pathway modulators. The screening methods of the invention may be used to identify substances having insecticidal properties and potential utility as insecticides or other pesticides, including but not limited to herbicides, nematicides and other worming agents, molluscicides, piscicides, etc. Optionally, the screening methods may further comprise conducting confirmatory tests on substances indicated to be insecticidal or potentially insecticidal based on the evaluation.

Parameters should be assessed less than 24 hours after contacting the insect cell with the insect hormone and/or GPCR signaling pathway modulator, so that evaluation is made while protein expression is induced. In some embodiments, the evaluation is made between 3 hours and up to 23 hours after initiating contact between the insect cell and the insect hormone and/or GPCR signaling pathway modulator. In some embodiments, the evaluation is made between 3 hours and 18 hours after initiating contact between the insect cell and the insect hormone and/or GPCR signaling pathway modulator. In some embodiments, the evaluation is made between 3 hours and 12 hours after initiating contact between the insect cell and the insect hormone and/or GPCR signaling pathway modulator. In some embodiments, the evaluation is made between 3 hours and 9 hours after initiating contact between the insect cell and the insect hormone and/or GPCR signaling pathway modulator. In some embodiments, the evaluation is made between 3 hours and 6 hours after initiating contact between the insect cell and the insect hormone and/or GPCR signaling pathway modulator. In some embodiments, the evaluation is made at about 1, 2, 3, 4, 5, 6, 7, 8, 9, 10, 11, 12, 13, 14, 15, 16, 17, 18, 19, 20, 21, 22, or 23 hours after initiating contact between the insect cell and the insect hormone and/or GPCR signaling pathway modulator.

The screening methods may further comprise identifying or categorizing the substance as insecticidal or potentially insecticidal based on the outcome of the evaluation, or as having some other effect on the insect cells or on the products of the insect cell. The screening methods may further comprise manufacturing substances found to be insecticidal or found to have some other desired effect on the insect cell.

In some embodiments, to prepare for an assay, the user fills each well of a plate with a candidate substance that the user wishes to conduct the experiment upon. The plate is a small container, usually disposable and made of plastic, which features a grid of small, open divots called wells.

After some incubation time has passed between the insect cells and the insect hormone or GPCR signaling pathway modulator (less than 24 hours), the candidate substance is added to allow the substance to absorb, bind to, or otherwise react (or fail to react) with the insect cells in the wells, and measurements are taken across all the plate's wells, either manually or by a machine. In an alternative embodiment, the substance is added to the insect cells before or simultaneously with the insect hormone or GPCR signaling pathway modulator. In an alternative embodiment, the candidate substances are in the wells, and the insect cells are added.

The screening methods may be done manually or may be high-throughput (HTS) and utilize automated liquid handling and plate manipulation. Automation is an important element in HTS's usefulness. Typically, an integrated robot system including one or more robots transports assay microplates from station to station for sample and reagent addition, mixing, incubation, and finally readout or detection.

Insect cells or candidate substances to be screened may be arrayed in a predetermined arrangement on multi-well plates (e.g., 96, 384, 1536, or more wells per plate). Multi-platform plate readers may be utilized. Screening large numbers of substances across a panel of targets produces a number of “active hits”. These “actives” can then be interrogated in much finer detail through secondary hit validation and selection of potentially potent agents for progression to much more in-depth study. Screening a wide range of substances in such a way can provide detailed information into the interaction of biological processes.

In some embodiments of the cells, compositions, and methods of the invention, the one or more proteins comprise one or more insecticide target proteins. For example, the proteins may be an ion channel (e.g., Kv2 potassium channel or neurotransmitter receptor).

In some embodiments of the cells, compositions, and methods of the invention, the insect cell is not a genetically modified cell (e.g., not a genetically engineered cell). Alternatively, the insect cell may be a genetically modified cell if desired. In some embodiments, the insect cell has been genetically modified to produce a protein that is not endogenous to the cell, or to increase production of an endogenous protein. In these embodiments, the endogenous or exogenous protein may or may not be induced by the hormone or GPCR signaling pathway modulator.

There are many varieties of insects from which cells may be obtained to induce protein expression. Cells from any insect of interest may be used in the practice of the invention. Further, the particular tissue of origin of the cell of the insect is not crucial. Culture medium may be used to sustain the cells. The culture medium that is employed may vary depending, for example, on the type of insect cell that is being induced. The medium may be supplemented as needed. Those of skill in the art are, in general, well acquainted with suitable insect cell culture techniques.

In some embodiments of the cells, compositions, and methods of the invention, the insect cell is a cell of a cell line. In some embodiments of the cells, compositions, and methods of the invention, the cells are Sua1B cells, Schneider 2 (S2) cells, BTI-TN-5B1-4 cells, Sf21 (IPLB-Sf21AE) cells, or SD cells. Cell lines can be obtained from various commercial sources or depositories such as the American Type Culture Collection. In other embodiments of the cells, compositions, and methods of the invention, the insect cell is a primary cell (an insect cell of a primary insect cell culture).

For example, in some embodiments, the cells are Anopheles gambiae cells. In some embodiments, the insect cells are Drosophila cells. In some embodiments, the insect cells are Trichoplusia ni cells. In some embodiments, the cells are Spodoptera frugiperda cells.

The insect cells can range in plasticity from totipotent or pluripotent stem cells (e.g., adult or embryonic), precursor or progenitor cells, to differentiated cells such as highly specialized cells of the central nervous system.

The one or more insect hormones or GPCR signaling pathway modulators used to produce the cells and compositions, and to practice the methods of the invention may be naturally occurring or artificial hormones or modulators, and may be obtained using methods known in the art, such by chemical synthesis or recombinant production.

In some embodiments of the cells, compositions, and methods of the invention, the insect hormone is selected from among adipokinetic hormone, allatostatin, bursicon, ecdysone, insect diuretic hormone, juvenile hormone (JH), prothoracic gland hormone, prothoracicotropic hormone (PTTH), vitellogenin, and insulin. In some embodiments, the insect hormone is an insect steroidal hormone having a four-ring steroid system. In some embodiments, the insect hormone is a tissue molting hormone (e.g., 20-hydroxyecdysone or other ecdysone receptor agonist, e.g., tebufenozide).

In some embodiments of the cells, compositions, and methods of the invention, the GPCR signaling pathway modulator is caffeine, or a caffeine derivative. A modulator of the GPCR signaling pathway induces protein expression and, without being limited by theory, inhibits the phosphodiesterase that hydrolyzes cyclic AMP, a product of GPCR activation, or acts upstream or downstream of phosphodiesterase to give a similar effect on protein expression. The GPCR signaling pathway modulator may act through one or more members of the pathway. For example, a GPCR signaling pathway modulator may act on the GPCR, adenylate cyclase, cAMP, or kinase to affect protein expression.

The insect hormone or GPCR signaling pathway modulator may be naturally occurring or non-naturally occurring.

One or more insect hormones or GPCR signaling pathway modulators may be contacted with the insect cell in an amount effective to induce protein expression (causing an increased amount of expression compared to the expression that occurs in the absence of the hormones or modulators). Protein expression may be increased, for example, 2-fold, 3-fold, 4-fold, 5-fold, 10-fold, or more. The amount of insect hormone or GPCR signaling pathway modulator used is that amount effective to induce protein expression. For example, the amount may be in the range of about 1 to about 100 μg/ml, or in the range of about 10 to about 20 μg/ml.

In some embodiments of the cells, compositions, and methods of the invention, the one or more insect hormones comprise a tissue molting hormone (e.g., 20-hydroxyecdysone).

The one or more proteins may be, for example, a secreted protein, membrane protein, or intracellular protein.

Optionally, the protein expression and screening methods may further include a step of purifying or isolating the one or more expressed proteins.

Optionally, the protein expression and screening methods may further include a step of characterizing the one or more proteins for which expression was induced by the hormone or GPCR signaling modulator (e.g., characterizing the proteins as insecticide target proteins or proteins that are not insecticide target proteins).

As used herein, the term “insect cell” encompasses a singular cell and a plurality of cells.

As used herein, the term “contacting”, “contact”, and grammatical variations thereof means bringing together two or more entities to make contact, regardless of which of the contacting entities are in motion. Thus, “contacting (a) with (b)” encompasses moving (a) into contact with (b), or moving (b) into contact with (a), or both. For example, the term “contacting” in the context of cells and hormones or GPCR signaling modulators is intended to mean bringing the insect cell and the one or more insect hormones and/or GPCR signaling modulators into contact, whether the cell is physically moved into contact with the hormone or GPCR signaling modulator, or vice-versa, or the insect cell and hormone or GPCR signaling modulator are both moved into contact with each other.

In the cells, compositions, and methods, the insect cells and an effective amount of one or more insect hormones and/or GPCR signaling pathway modulators are brought into contact with each other for a sufficient duration of time to induce the expression of one or more proteins. In some embodiments, the duration is less than about 24 hours. In some embodiments, the duration is about 1 hour to about 3 hours. In some embodiments, the duration is about 3 hours to about 6 hours. In some embodiments, the duration of time is about 1, 2, 3, 4, 5, 6, 7, 8, 9, 10, 11, 12, 13, 14, 15, 16, 17, 18, 19, 20, 21, 22, or 23 hours.

As used herein, the term “substance” in the context of the screening methods of the invention is intended broadly to include any substance for which an effect on the cell or on its products are to be evaluated. For example, the substance may be a chemical composition (e.g., small molecule), or biological product (a biologic), such as a protein or nucleic acid, other cells, natural or synthetic polymers, toxins, etc. The substance may be in any physical state (liquid, solid, gas, etc.). The substance may naturally occurring or non-naturally occurring. The substance may be a type of energy, such as radiation. Specific examples of substances that may be screened using the screening methods of the invention include, but are not limited to, acetylcholinesterase inhibitors (e.g., carbamates and organophosphates), GABA-gated chloride channel antagonists (e.g., fipronil and endosulfan), sodium channel modulators (e.g., pyrethroids, or veratridine and related compounds), nicotinic acetylcholine receptor agonists (e.g., neonicotinoids such as imidacloprid), nicotinic acetylcholine receptor allosteric activators (e.g., spinosyns), nicotinic acetylcholine receptor channel blockers (e.g., cartap and bensultap), chloride channel activators (e.g., avermectins), microbial membrane disruptors (toxins from Bacillus thuringiensis or Bacillus sphaericus), mitochondrial poisons (e.g., diafenthiuron, chlorfenapyr, hydramethylnon, fenazaquin, etc.), octopamine receptor agonists (e.g., amitraz), voltage-dependent sodium channel blockers (e.g., indoxacarb), ryanodine receptor modulators (e.g., chlorantraniliprole), muscarinic agonists (e.g., bethanechol, pilocarpine) etc.

The insect cells, substances, insect hormones, and GPCR signaling pathway modulator may be placed in combination with a carrier, which may naturally occurring or non-naturally occurring.

Various insect cell culture and insect cell screening methods may be used with the cells, compositions, and methods of the invention (see, e.g., [4]-[8], which are incorporated herein by reference in their entirety).

### Exemplified Embodiments

Embodiment 1: A screening method, comprising: providing an insect cell, wherein the insect cell has been contacted with an effective amount of one or more insect hormones or modulators of the G protein-coupled receptor signaling pathway (GPCR signaling pathway modulators) to express one or more proteins; contacting the insect cell with a substance; and evaluating one or more parameters to determine the effect of the substance on the insect cell.

Embodiment 2: The screening method of embodiment 1, wherein said evaluating is conducted at less than 24 hours after initiation of contact with the insect hormone or GPCR signaling pathway modulator.

Embodiment 3: The screening method of embodiment 1, wherein said evaluating is conducted between 3 hours and 12 hours after initiation of contact with the insect hormone or GPCR signaling pathway modulator.

Embodiment 4: A screening method, comprising contacting an insect cell with an effective amount of one or more insect hormones or modulators of the G protein-coupled receptor signaling pathway (GPCR signaling modulators); contacting the insect cell with a substance before, during, or after contact with the one or more insect hormones or GPCR signaling pathway modulators; and evaluating one or more parameters to determine the effect of the substance on the insect cell.

Embodiment 5: The screening method of embodiment 4, wherein the evaluating is conducted at less than 24 hours after initiation of contact with the insect hormone or GPCR signaling pathway modulator.

Embodiment 6: The screening method of embodiment 1, wherein said evaluating is conducted between 3 hours and 12 hours after initiation of contact with the insect hormone or GPCR signaling pathway modulator.

Embodiment 7: The method of any one of embodiments 4-6, wherein said evaluating comprises measuring the function of one or more ion channels before, during, and/or after contact with the substance and/or before, during, and/or after contact with the one or more insect hormones or GPCR signaling pathway modulators.

Embodiment 8: The method of embodiment 1 or 4, wherein the substance is a small molecule, protein, or nucleic acid.

Embodiment 9: The method of embodiment 1 or 4, wherein the substance is a acetylcholinesterase inhibitor, GABA-gated chloride channel antagonist, sodium channel modulator, nicotinic acetylcholine receptor agonist, nicotinic acetylcholine receptor allosteric activator, nicotinic acetylcholine receptor channel blockers, chloride channel activator, microbial membrane disruptor, mitochondrial poison, octopamine receptor agonist, voltage-dependent sodium channel blocker, ryanodine receptor modulator, or two or more of the foregoing.

Embodiment 10: The method of embodiment 1 or 4, wherein the insect cell is contacted with the insect hormone, and the insect hormone is selected from among adipokinetic hormone, allatostatin, bursicon, ecdysone, insect diuretic hormone, juvenile hormone (JH), prothoracic gland hormone, prothoracicotropic hormone (PTTH), insulin, and vitellogenin.

Embodiment 11: The method of embodiment 1 or 4, wherein the insect cell is contacted with the insect hormone, and wherein the insect hormone comprises tissue molting hormone (e.g., 20-hydroxyecdysone or other ecdysone receptor agonist, e.g., tebufenozide).

Embodiment 12: The method of embodiment 1 or 4, wherein the insect cell is contacted with the GPCR signaling pathway modulator, and wherein the GPCR signaling pathway modulator is caffeine, or a caffeine derivative.

Embodiment 13. A method for inducing protein expression in insect cells, comprising contacting an insect cell with an effective amount of one or more insect hormones or modulators of the G protein-coupled receptor signaling pathway (GPCR signaling pathway modulators) to induce expression of one or more proteins.

Embodiment 14: The method of embodiment 13, wherein the one or more proteins comprise one or more insecticide target proteins.

Embodiment 15: The method of embodiment 13 or 14, wherein the insect cell is in cell culture with a culture medium, and wherein the one or more insect hormones or GPCR signaling pathway modulators are added to the culture medium (e.g., growth media).

Embodiment 16: The method of any one of embodiments 13-15, wherein the insect cell is not a genetically modified cell.

Embodiment 17: The method of any one of embodiments 13-15, wherein the insect cell is a genetically modified cell. Embodiment 18: The method of any one of embodiments 13-17, wherein the insect cell is a plurality of insect cells.

Embodiment 19: The method of one of embodiments 13-18, wherein the insect cell is a cell line.

Embodiment 20: The method of embodiment 19, wherein the insect cell is a cell line selected from among Sua1B cells, Schneider 2 (S2) cells, BTI-TN-5B1-4 cells, Sf21 (IPLB-Sf21AE) cells, and 519 cells.

Embodiment 21: The method of embodiment 20, wherein the insect cell is Sua1B cells.

Embodiment 22: The method of any one of embodiments 13-21, wherein the one or more insect hormones comprises a naturally occurring hormone.

Embodiment 23: The method of any one of embodiments 13-21, wherein the one or more insect hormones comprises an artificial hormone.

Embodiment 24: The method of any one of embodiments 13-23, wherein the insect cell is contacted with the insect hormone, and wherein the insect hormone is selected from among adipokinetic hormone, allatostatin, bursicon, ecdysone, insect diuretic hormone, juvenile hormone (JH), prothoracic gland hormone, prothoracicotropic hormone (PTTH), insulin, and vitellogenin.

Embodiment 25: The method of any one of embodiments 13-23, wherein the insect cell is contacted with the insect hormone, and wherein the insect hormone comprises tissue molting hormone (e.g., 20-hydroxyecdysone).

Embodiment 26: The method of any one of embodiments 13-21, wherein the insect cell is contacted with the GPCR signaling pathway inhibitor, and wherein the GPCR signaling pathway modulator is caffeine, a caffeine derivative, or a molecule acting elsewhere in the pathway.

Embodiment 27: The method of embodiment 13, wherein the one or more proteins is a secreted protein, membrane protein, or intracellular protein.

Embodiment 28: The method of embodiment 13, further comprising purifying or isolating the one or more expressed proteins.

Embodiment 29: The method of any one of embodiments 13-29, further comprising measuring the expression of the one more proteins less than 24 hours after initiating said contacting.

Embodiment 30: The method of any one of embodiments 13 - 28, further comprising measuring the expression of the one or more proteins between 3 hours and 12 hours after initiating said contacting.

Embodiment 31: An insect cell that has been induced to express one or more proteins by contact with one or more insect hormones or modulators of G protein-coupled receptor signaling (GPCR signaling pathway modulators).

Embodiment 32: A composition comprising an insect cell of embodiment 31. Embodiment 33: A composition comprising an insect cell and one or more insect hormones or modulators of the G protein-coupled receptor signaling pathway (GPCR signaling pathway modulators).

Embodiment 34: The composition of embodiment 33, wherein the insect cell has been induced to express one or more proteins by the one or more insect hormones or modulators of the G protein-coupled receptor signaling pathway (GPCR signaling pathway modulators). Embodiment 35: The composition of any one of embodiments 32 - 34, wherein the composition is a cell culture.

Embodiment 36: The composition of any one of embodiments 32 - 35, further comprising a carrier.

Embodiment 37: The composition of embodiment 36, wherein the carrier is naturally occurring.

Embodiment 38: The composition of embodiment 36, wherein the carrier is not naturally occurring.

Embodiment 39: The composition of any one of embodiments 32 to 38, wherein the insect hormone or GPCR signaling pathway modulator is naturally occurring.

Embodiment 40: The composition of any one of embodiments 32 to 38, wherein the insect hormone or GPCR signaling pathway modulator is non-naturally occurring.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### MATERIALS AND METHODS

Cell Culture. Sua1B insect cells were maintained in tissue culture flasks with Schneider's insect media supplemented with 10% fetal bovine serum (FBS) and 100 U/mL penicillin and streptomycin. The culture was maintained at 28° C. in an incubator without CO₂ amendment. Cells were passed every 3-5 days.

Human embryonic kidney (HEK)-293 cells (CRL-1573) were maintained on Dulbecco's modified Eagle's medium (DMEM) supplemented with 15% FBS. Engineered cells with Anopheles gambiae Kv2 (AgKv2) channel gene (Accession # XM_315955.4) had a CMV promoter and tetracycline regulation for expression. The cell line was engineered by Dualsystems Biotech AG (Switzerland).

Differentiation with 20-Hydroxyecdysone. 20-HE was added immediately after maintenance (24 hr experiments) or to fresh growth media for cells grown in dishes (electrophysiology) or 96 well plates (fluorescence) 3 hours before experiments were run. Controls had 0.1% DMSO.

Electrophysiology. Patch clamp recordings of potassium channel currents were performed using standard methods [4]. Cells were maintained at a holding potential of −80 mV, and stepped up to +100 mV in 10 mV increments each for 3 milliseconds. Each full trace ran for 1 second or was adjusted as needed for clear display.

Thallium Flux Measurements of Potassium Channel Pharmacology. The FluxOR™ potassium channel assay (Molecular Probes, Inc., Eugene, Oreg., USA) [3] was performed as outlined in the product information sheet and performed on the SyntaxMax plate reader (BioTek, Winooski, Vt., USA).

### EXAMPLE 1—Voltage-Sensitive Potassium Channels Expressed by Hormone Treatment in Mosquito Cell lines

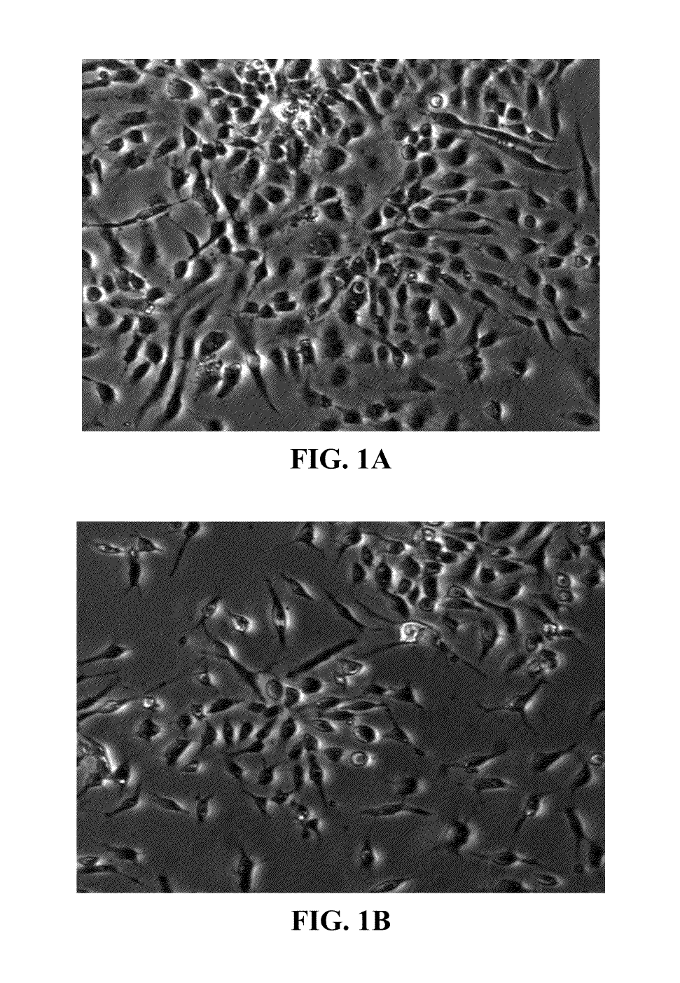

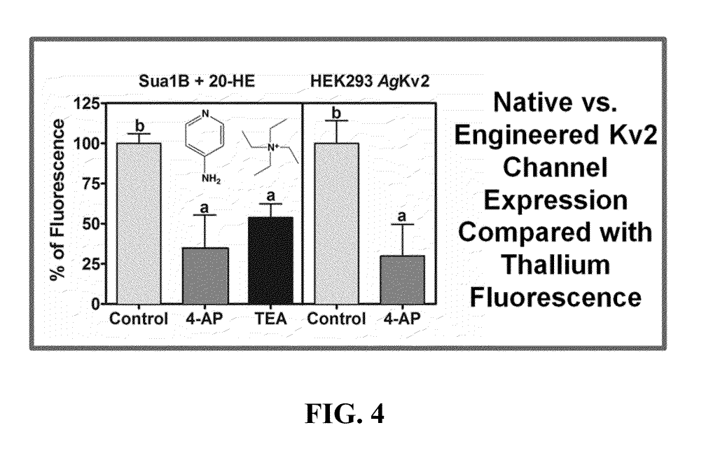

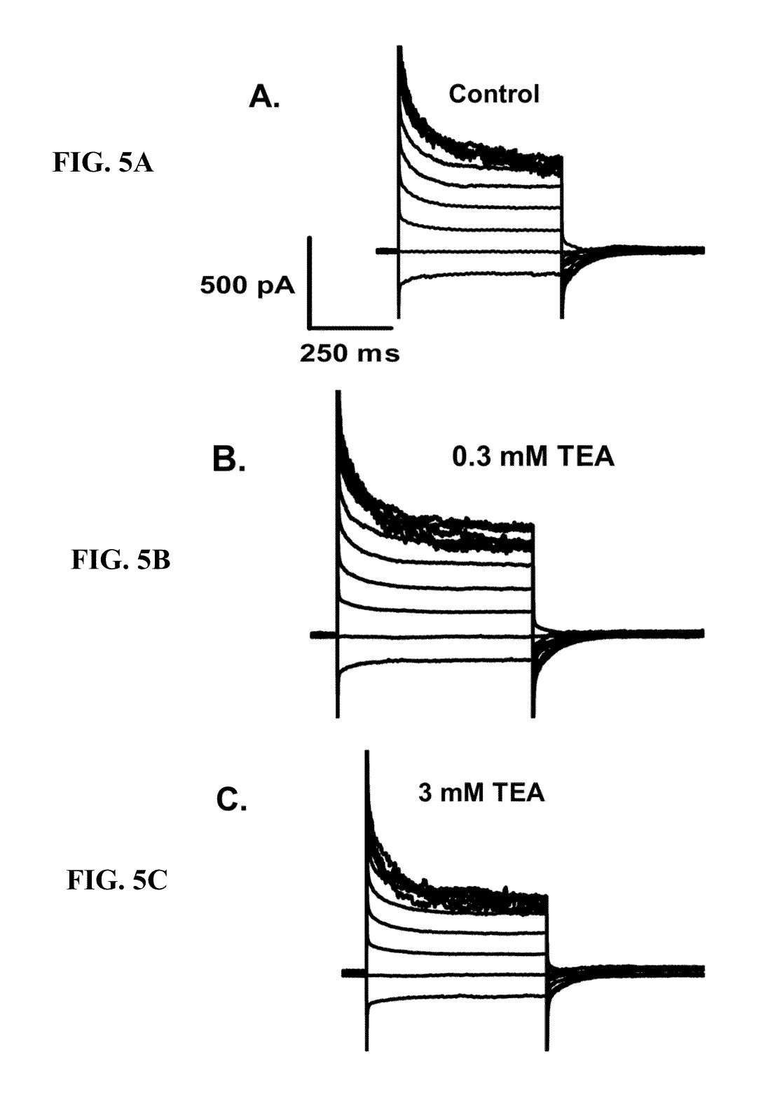

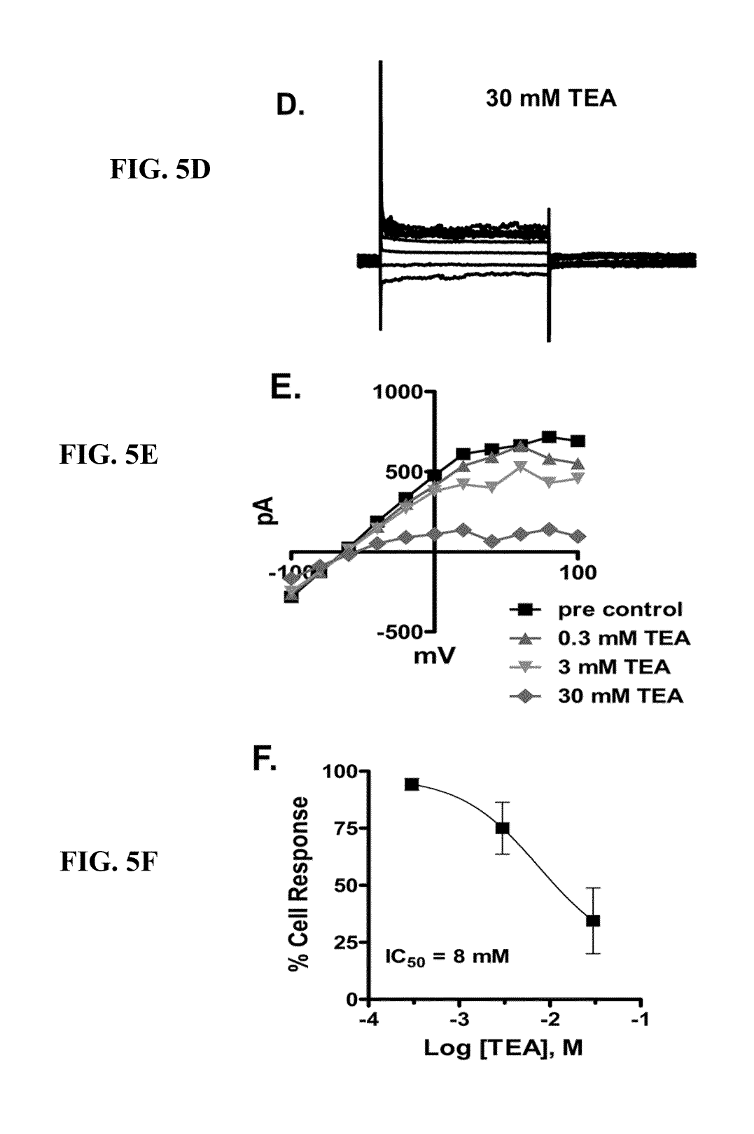

The goal of these experiments was to evaluate the presence of insecticide target proteins in hormone-induced differentiated insect cells, which could lead to new high-throughput screening (HTS) methods and a source of insect proteins for basic research. Results are shown in FIGS. 1A-B, 2, 3A-B, 4, 5A-F, and 6A-F. This study used cultures of Sua1B cells and 20-hydroxyecdysone (20-HE), an insect molting hormone [1], to initiate expression of insecticide target proteins and to evaluate toxicant effects. Whole cell patch clamp techniques show Kv2 delayed-rectifier potassium channels expressed in as little as 3 hours after treatment with 20-HE. The expressed currents had current-voltage relationships diagnostic for these channels, and were inhibited by 4-aminopyridine (4-AP) and tetraethylammonium (TEA), well-established potassium channel blockers having insecticidal properties [2], as shown in FIG. 4. The electrophysiological results were confirmed in the established thallium fluorescence assay of potassium channel function, which is used for HTS drug screening (Li Q. et al., “Identification of novel KCNQ4 openers by a high-throughput fluorescence-based thallium flux assay”, Anal Biochem, 2011, 418(1):66-72). The presence of ion channels and receptors in these cells will accelerate HTS for new insecticides, and make screening more economical.

The inventors determined that treatment of immortal insect cell lines with the insect hormone 20-hydroxyecdysone induces expression of insecticide target proteins (e.g., the Kv2 potassium channel) without the need for genetic engineering. These hormone-treated cell lines are ideal for high throughput screening applications.

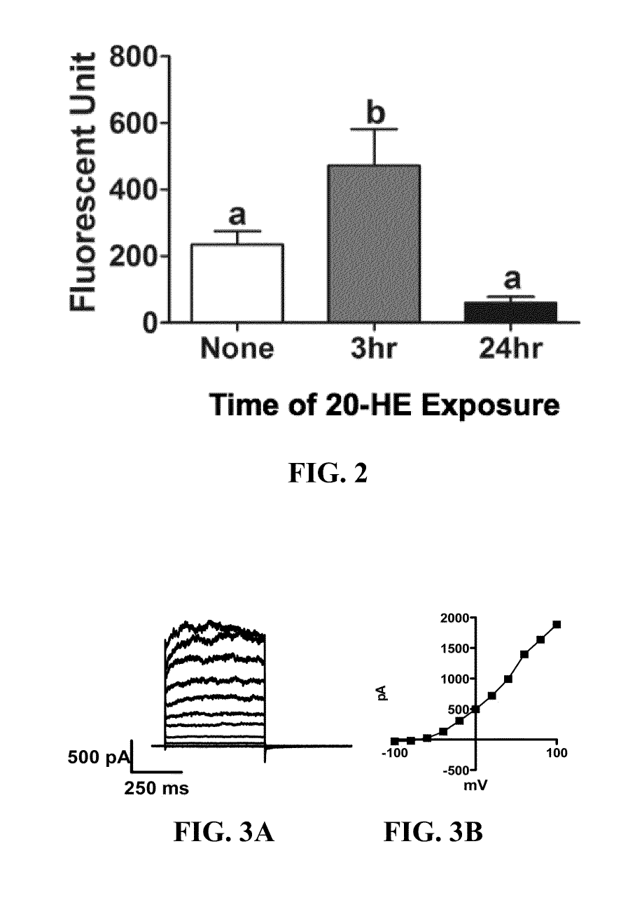

Treatment of 20-hydroxyecdysone (20-HE) to Sua1B cells for 24 hours inhibited cell growth (see FIGS. 1A and 1B), but caused detectable ion channel expression in hours (see FIG. 2). In the absence of 20-HE treatment, little or no K⁺ channel expressing cells can be identified (see FIG. 2).

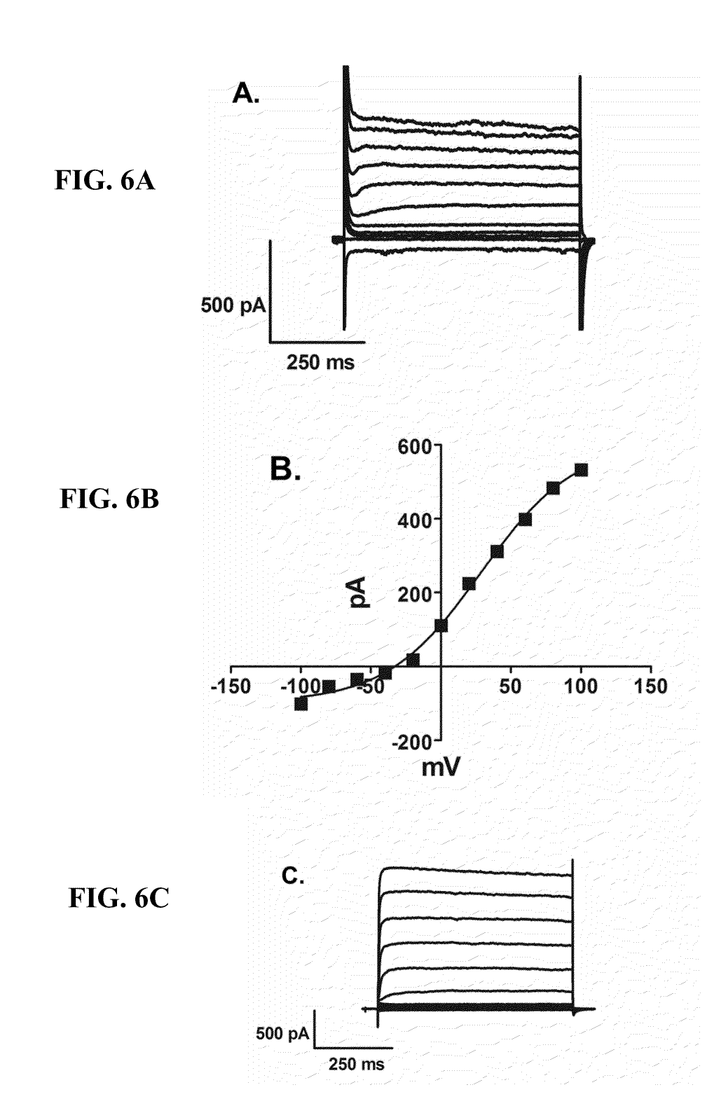

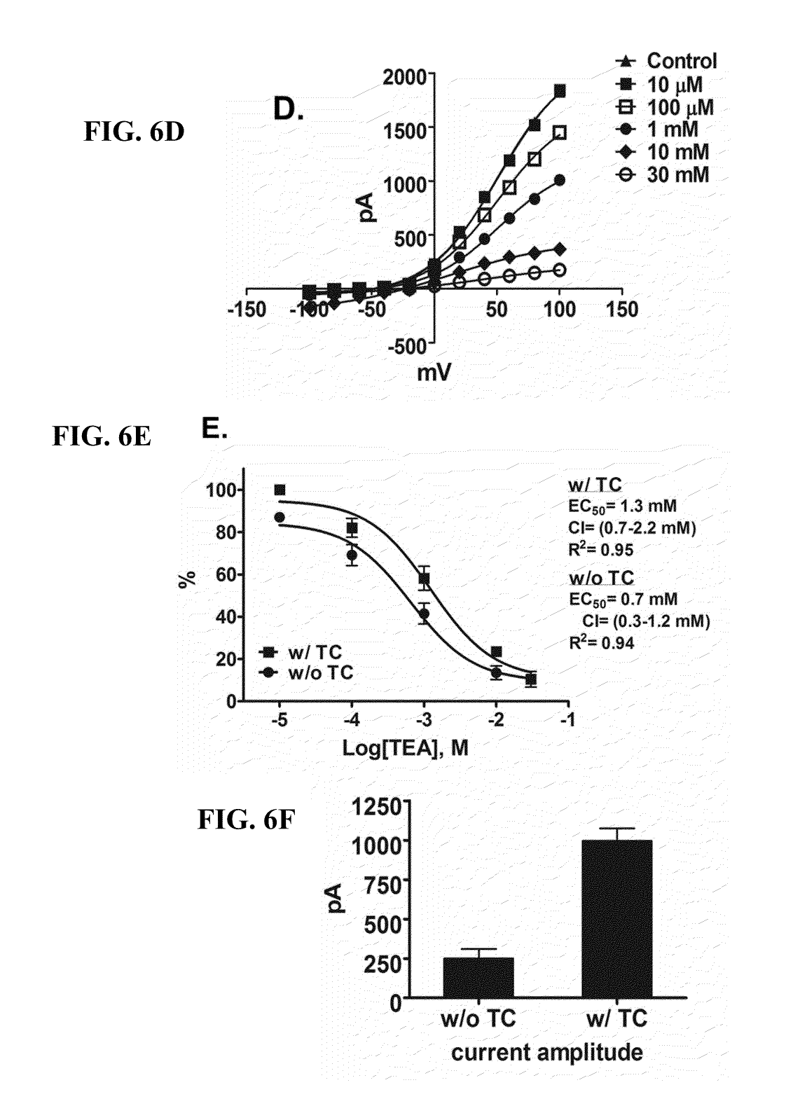

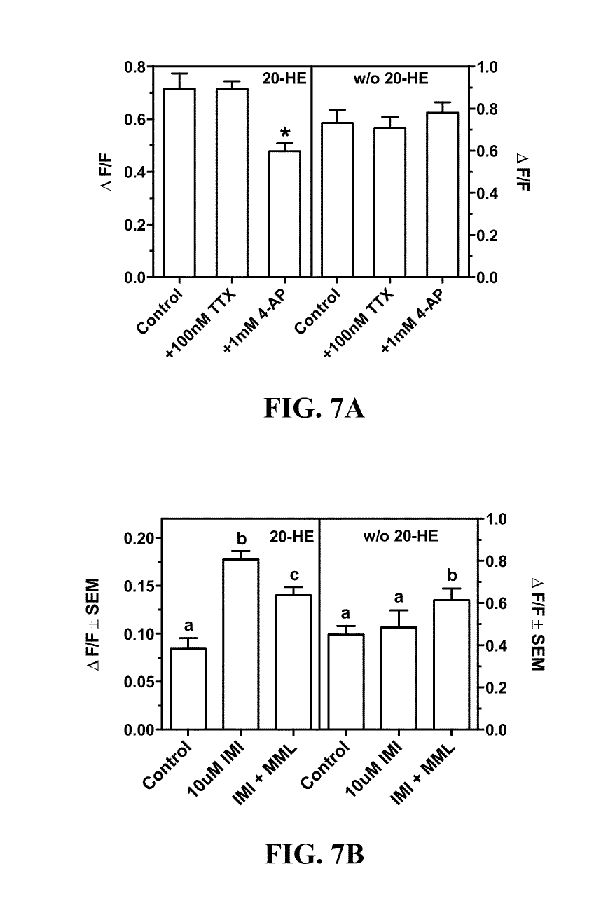

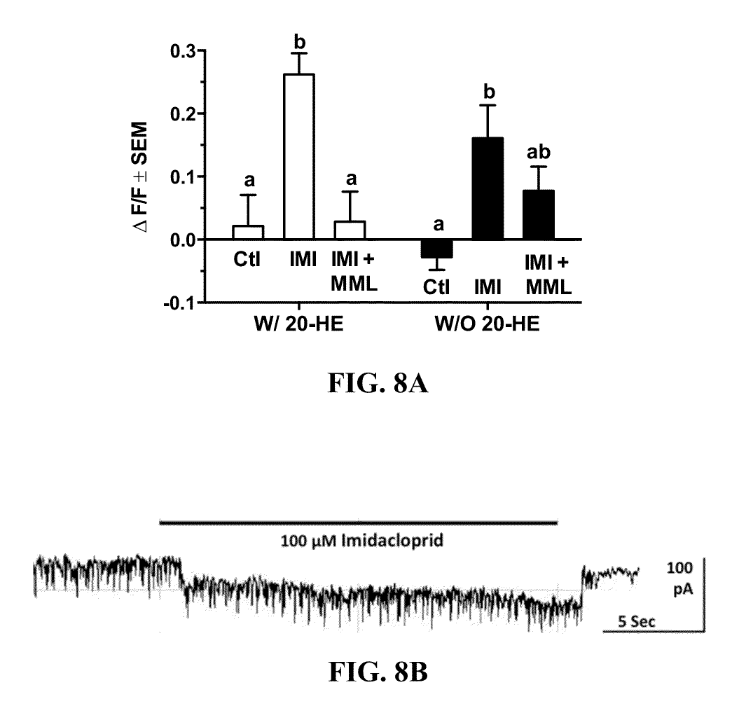

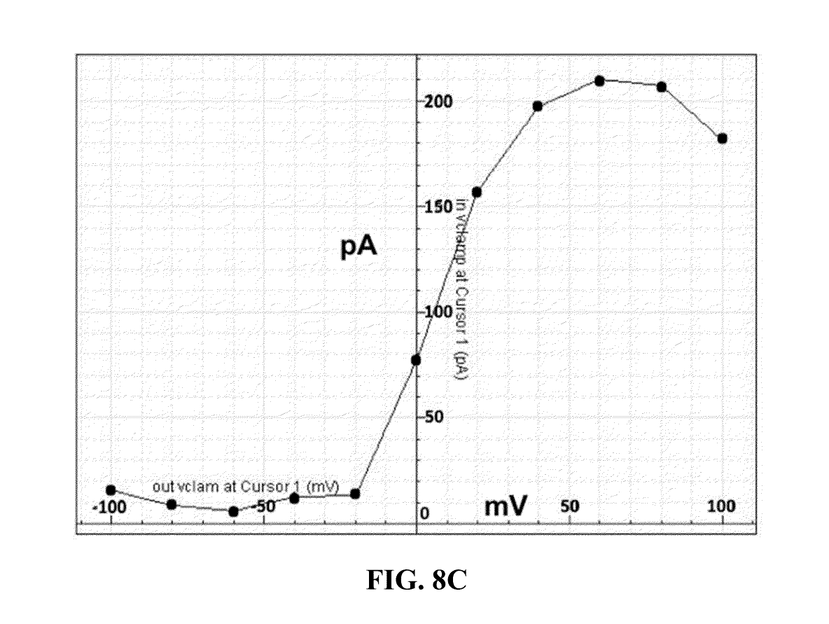

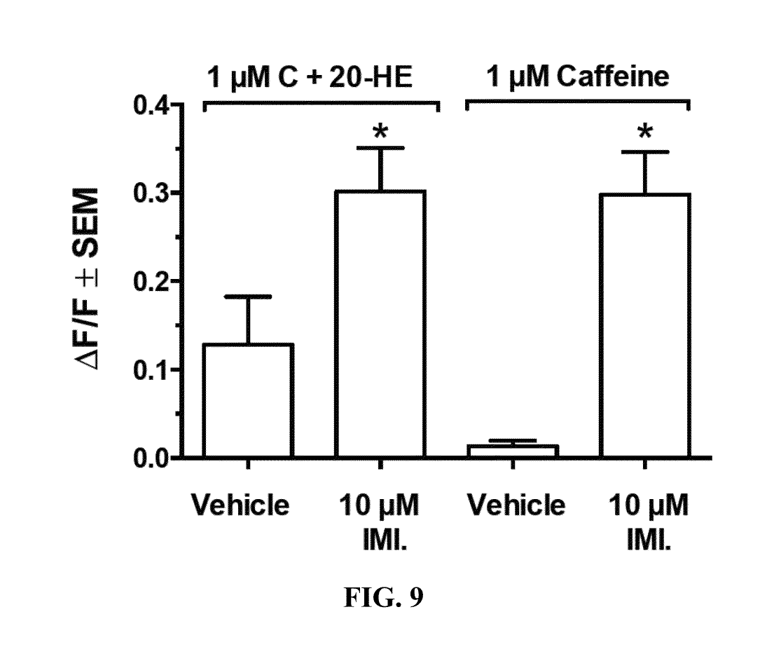

These results suggest that the expression of ion channels may have caused cell loss, which is why TC-sensitive promoters are often used in engineered cell lines. Maximal increase of AgKv2 channel expression by 20-HE treatment in Sua1B cells (2000 pA; see FIGS. 3A and 3B) is similar to that of genetically engineered HEK293 cells (1800 pA; see FIG. 6D). 4-AP and TEA, insecticidal potassium channel blockers, inhibited AgKv2 currents in Sua1B cells, similar to engineered AgKv2 in HEK293 cells. Results of further experiments are shown in FIGS. 7A and 7B, 8A-8C, and 9.

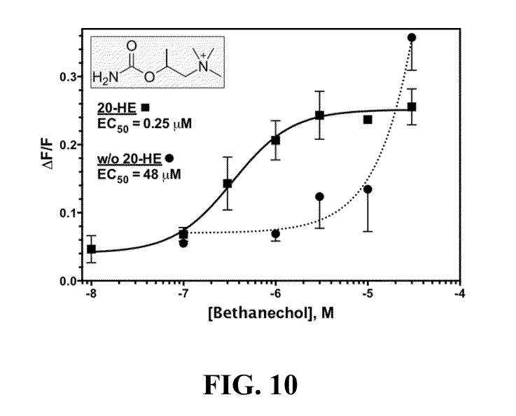

The muscarinic agonist, bethanechol, a mimic of acetylcholine that preferentially activates the muscarinic-type receptor, increased calcium fluorescence in Sua1B cells treated with the insect tissue molting hormone 20-HE. As shown in FIG. 10, the response was a typical sigmoid curve with an EC₅₀ (concentration where the response is half maximal) of 0.25 micromolar. In the absence of the hormone, bethanechol activated fluorescence, but had a less potent effect, with an EC₅₀ of 48 micromolar. Thus, the response in the absence of insect hormone is nearly 200-fold less potent, most likely indicating a different receptor or a kind of non-specific effect. Similar results were observed for pilocarpine, a specific muscarinic agonist. All these data support the concept that cell culture treatment with hormone induced expression of an acetylcholine muscarinic receptor agonist in insect cell cultures.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

[1] C. Ress, M. Holtmann, U. Maas, J. Sofsky, A. Dorn. 2000. Tissue and Cell. 200032(6): 464-477.

[2] N. R. Larson, B. Sun, P. Carlier, M. Ma, and J. R. Bloomquist. 2013. Evaluation of synthetic compounds as novel mosquitocides targeting potassium channels for control of Aedes aegypti and Anopheles gambiae. National Meeting of the American Chemical Society, Indianapolis, Ind.

[3] http://www.lifetechnologies.com/order/catalog/product/F10017.

[4] Invitrogen (03/08/2010). “Cell Lines”. Growth and Maintenance of Insect cell lines. Rev. Date—8 March 2010. Invitrogen. Manual part no. 25-0127, MAN0000030. Retrieved Feb. 23, 2012.

[5] D. E. Lynn, “Development and characterization of insect cell lines”, Cytotechnology, 1996, Volume 20, Issue 1-3, pp. 3-11.

[6] D. E. Lynn, “Methods for Maintaining Insect Cell Cultures, Journal of Insect Science, May 20, 2002.

[7] Wickham T. J., “Screening of Insect Cell Lines for the Production of Recombinant Proteins and Infectious Virus in the Baculovirus Expression System”, Biotechnol. Prog., 1992, 8:391-396.

[8] Lynn, D. E., Development of insect cell lines: virus susceptibility and applicability to prawn cell culture. Methods in Cell Sci., 1999, 21(4):173-81.

We claim:

1. A screening method, comprising: providing an insect cell, wherein the insect cell has been contacted with an effective amount of one or more insect hormones or modulators of the G protein-coupled receptor signaling pathway (GPCR signaling pathway modulators) to express one or more proteins; contacting the insect cell with a substance; and evaluating one or more parameters to determine the effect of the substance on the insect cell.

2. A screening method, comprising contacting an insect cell with an effective amount of one or more insect hormones or modulators of the G protein-coupled receptor signaling pathway (GPCR signaling modulators); contacting the insect cell with a substance before, during, or after contact with the one or more insect hormones or GPCR signaling pathway modulators; and evaluating one or more parameters to determine the effect of the substance on the insect cell.

3. The screening method of claim 2, wherein the evaluating is conducted at less than 24 hours after initiation of contact with the insect hormone or GPCR signaling pathway modulator.

4. The screening method of claim 2, wherein said evaluating is conducted between 3 hours and 12 hours after initiation of contact with the insect hormone or GPCR signaling pathway modulator.

5. The method of 2, wherein said evaluating comprises measuring the function of one or more ion channels before, during, and/or after contact with the substance and/or before, during, and/or after contact with the one or more insect hormones or GPCR signaling pathway modulators.

6. The method of claim 2, wherein the substance is a acetylcholinesterase inhibitor, GABA-gated chloride channel antagonist, sodium channel modulator, nicotinic acetylcholine receptor agonist, nicotinic acetylcholine receptor allosteric activator, nicotinic acetylcholine receptor channel blockers, chloride channel activator, microbial membrane disruptor, mitochondrial poison, octopamine receptor agonist, voltage-dependent sodium channel blocker, ryanodine receptor modulator, or two or more of the foregoing.

7. The method of 2, wherein the insect cell is contacted with the insect hormone, and the insect hormone is selected from among adipokinetic hormone, allatostatin, bursicon, ecdysone, insect diuretic hormone, juvenile hormone (JH), prothoracic gland hormone, prothoracicotropic hormone (PTTH), insulin, vitellogenin, and tissue molting hormone.

8. A method for inducing protein expression in insect cells, comprising contacting an insect cell with an effective amount of one or more insect hormones or modulators of the G protein-coupled receptor signaling pathway (GPCR signaling pathway modulators) to induce expression of one or more proteins.

9. The method of claim 8, wherein the one or more proteins comprise one or more insecticide target proteins.

10. The method of claim 8, wherein the insect cell is in cell culture with a culture medium, and wherein the one or more insect hormones or GPCR signaling pathway modulators are added to the culture medium (e.g., growth media).

11. The method of claim 8, wherein the insect cell is not a genetically modified cell.

12. The method of claim 8, wherein the insect cell is a cell line selected from among Sua1B cells, Schneider 2 (S2) cells, BTI-TN-5B1-4 cells, Sf21 (IPLB-Sf21AE) cells, and Sf9 cells.

13. The method of claim 8, wherein the insect cell is contacted with the insect hormone, and wherein the insect hormone is selected from among adipokinetic hormone, allatostatin, bursicon, ecdysone, insect diuretic hormone, juvenile hormone (JH), prothoracic gland hormone, prothoracicotropic hormone (PTTH), insulin, vitellogenin, and tissue molting hormone.

14. The method of claim 8, further comprising purifying or isolating the one or more expressed proteins.

15. The method of claim 8, further comprising measuring the expression of the one more proteins less than 24 hours after initiating said contacting.

16. The method of claim 8, further comprising measuring the expression of the one or more proteins between 3 hours and 12 hours after initiating said contacting.

17. An insect cell that has been induced to express one or more proteins by contact with one or more insect hormones or modulators of G protein-coupled receptor signaling (GPCR signaling pathway modulators).

18. A composition comprising an insect cell of claim 17.

19. A composition comprising an insect cell and one or more insect hormones or modulators of the G protein-coupled receptor signaling pathway (GPCR signaling pathway modulators).

20. The composition of claim 19, wherein the insect cell has been induced to express one or more proteins by the one or more insect hormones or modulators of the G protein-coupled receptor signaling pathway (GPCR signaling pathway modulators).
